(19) United States
(12) Patent Application Publication
(10) Pub. No.: US 2024/0181116 A1
(43) Pub. Date: Jun. 6, 2024

(54) AIR STERILIZATION DEVICE, SYSTEM AND METHOD FOR PLUMBING VENTILATING PIPE

(71) Applicants: Ronald Chun Yu Lam, Hong Kong (CN); Louis Chi Hung Lam, Hong Kong (CN)

(72) Inventors: Ronald Chun Yu Lam, Hong Kong (CN); Louis Chi Hung Lam, Hong Kong (CN)

(21) Appl. No.: 18/554,827

(22) PCT Filed: Feb. 16, 2022
(86) PCT No.: PCT/CA2022/050222

§ 371 Date: Oct. 11, 2023

Related U.S. Application Data
(60) Provisional application No. 63/151,816, filed on Feb. 22, 2021.

(51) Int. Cl. A61L 9/20; A61L 9/22

(52) U.S. Cl. CPC A61L 9/20; A61L 9/22; A61L 2209/111; A61L 2209/12

(57) ABSTRACT

An air sterilization device for plumbing air ventilation piping and a system therefor with sensors, network connectivity, monitoring and controlling. The air sterilization device is disposed coaxially with a plumbing ventilating pipe to ensure air escaping out from the ventilating pipe has been sterilized.

### CROSS-REFERENCE TO RELATED APPLICATIONS

The present application claims priority to U.S. Provisional Application No. 63/151,816, filed on Feb. 22, 2021, the contents of which are incorporated herein by reference in its entirety.

### STATEMENT REGARDING FEDERALLY SPONSORED RESEARCH OR DEVELOPMENT

Not Applicable.

### NAMES OF THE PARTIES TO A JOINT RESEARCH AGREEMENT

Not Applicable.

### BACKGROUND OF THE INVENTION

### Field of the Invention

The present invention generally relates to an air sterilization device for plumbing air ventilating piping. The present invention further relates to an air sterilization device for plumbing air ventilation with sensors, network connectivity, monitoring and control. The air sterilization device is installed coaxially with a plumbing ventilating pipe, acting as an anti-siphonage pipe, of a plumbing system to ensure air escaping out from the ventilating pipe has been sterilized.

The present invention yet further relates to a control system that, via communication means, ensures application of optimal amount of sterilization energy by the air sterilization device based on meteorological conditions at the location where the air sterilization device is installed.

The present invention yet further relates to a control system that, manages the utilization of sterilization elements within the air sterilization device to maximize their usage before replacement and to ensure the device's maintenance is conducted in a timely manner to ensure a fully continuous operational air sterilization device.

### Description of Related Art

Plumbing systems are essential for modern civilization. Within a plumbing system, ventilating pipes or anti-siphonage pipes are critical for smooth flow of effluents, providing pressure equalization function within the plumbing system. In densely populated urban areas, with high-rise buildings constructed, ventilating pipes will likely service multiple facilities aligned vertically. The multi-connections to a single pipe, though simplifies the plumbing system construction, may or could, sometime, create unexpected consequences. From various studies and researches, it was found that some viruses and/or some pathogens may be shed from the human body via waste. The shed viruses and pathogen particles may travel through the plumbing pipes and could attach to droplets and/or aerosols inside the sewer and ventilating pipe systems. Subsequently, the virus and/or pathogens could traverse various piping systems with air movements within the piping systems.

Of particular concern is emergent virus, like the Severe Acute Respiratory Syndrome Coronavirus 2, SAR-Cov-2 (or COVID19). It was found that the virus also shed as it infects the human host. Once the shed virus enters into the sewage plumbing system, the sewage and ventilating pipes may/would harbor the virus contaminated particles and particulate matters.

In order for plumbing ventilating pipes to provide pressure equalization or release of sewer gas within a plumbing/sewage system, plumbing ventilating pipes are exposed to the outside environment to draw in air/to exhaust sewer gas. Typically, one end is raised to the rooftop of a building and vented to outside air via roof vent. In normal operations, the air is typically vented through a roof vent and equalizes pressure as water traverse down the plumbing or sewage pipes. However, with steady wind flowing across the roof, negative pressure may develop atop the ventilating pipe or about the roof vent. This negative pressure will cause air inside the ventilating pipes to move out from the opening of the roof vent. Recent studies in Hong Kong have confirmed that COVID19 virus traces were found on the rooftops of a number of buildings around their roof vents of the sewage system serving residential units with residents infected with the virus.

In view of the large number of ventilating and sewage pipes in an urban environment, the probability of spreading of a disease-causing virus and/or pathogen via plumbing/sewage ventilating piping can be very high. Ensuring air escaping from the top of the ventilating pipes is free from pathogens is of high importance. Applying appropriate techniques and technologies to inactivate pathogens coming out of the ventilating pipes without affecting the operation of a plumbing system is eminently required.

Ultraviolet light in the C-band wavelength (or UVC) has been shown to be highly effective in inactivating pathogens, including various viruses. Laboratory studies showed that, depending on the length of the ventilating pipe and the speed of the wind blowing about the vent opening, the speed of air moving out therefrom can be very high. Accordingly, not only its direction of the air flowing into or out from the vent opening, but also the speed of the air flow through the vent opening would vary significantly over a time period.

Taking energy consumption and carbon emission into consideration, a sterilization system for inactivating pathogens in a ventilating or anti-siphoning pipe should also be energized judiciously. The ideal inactivation system should be designed in such a way that, when the wind in the environment is gentle and not likely to generate significant low pressure at the top of the pipe, the inactivation power can be reduced to save energy. Should the wind across the pipe be more significant, the system should provide higher power for inactivation. Furthermore, given the critical nature of the sterilization device, ultraviolet light source should be monitored to ensure that the device stand ready for any sterilization need. The system should be able to indicate when elements within the system are to be replaced. Due to the damaging nature of UVC to the human body, care must be given to the maintenance personnel. The sterilization system should remotely notify maintenance personnel the time at which service is required.

Various attempts have been made and proposed previously for using UVC for sterilization. For example, CN patent No. 203898790 to Gao, contents of which is incorporated herein by reference, relates to sterilization of air within a pipe utilizing UVC within a pipe; however, the device is for medical use only and require additional glass or transparent tubing.

CN patent application CN 105999352 A to Hua Dong, contents of which is incorporated herein by reference, discloses a device utilizing UVC to sterilize air from a ventilating pipe, while ensuring barriers are placed in the path of air flow; however, the barrier restricts and reduces air flow for the ventilating pipe and does not account for maintenance and efficacy of the device.

CN patent application CN 111256243 A to Si Ping, contents of which is incorporated herein by reference, discloses a system utilizing UVC LED to sterilize air within a ventilation system and utilizes controls to notify users.

There are, at least, for example, three major shortcomings in the prior attempts.

Firstly, air flow of the ventilating pipe is restricted in the described teachings of the prior arts. The air within the pipe is diverted to increase travel time. However, for a ventilating pipe, the anti-siphonage application requires air flow as unrestricted as possible.

Secondly, the control system in the described teachings does not take into account of external environmental conditions under various wind conditions. For a ventilating pipe, the wind speed near the sterilization device will greatly affect the sterilization energy required.

Thirdly, the control system in the described teachings does not proactively provide maintenance schedule and guidelines based on the utilization and intensity required for the sterilization of the air within the ventilating pipe. For sterilization purposes, failure of the sterilization elements unnoticed would defeat the purpose of the device, thus maintenance should be conducted proactively but in a targeted manner to reduce cost.

Accordingly, there is a long-felt but urgent need to develop an air sterilization device that can sterilize air moving within existing plumbing/swage ventilating pipes, provide connectivity to manage the power utilized and capture potential savings and leverage the connectivity to provide timely maintenance and servicing to the device.

### BRIEF SUMMARY OF THE INVENTION

An object of the present patent application is to provide an air sterilization system for ventilating pipes; the air sterilization system that utilizes ultraviolet-C (or UV-C) light/radiation emitter in the spectrum frequency between 200 nm to 280 nm.

Another object of the present patent application is to provide a sensors system to capture various information within the air sterilization system; the information can be retained in the air sterilization system or sent to a computation system for optimization and control.

Yet another object of the present patent application is to provide a wireless and/or wired communication system for communication with a computation system that monitor and control the air sterilization system.

Yet another object of the present patent application is to provide a computation system that stores and process the data from the air sterilization system, the computation system also provides control signals to the sterilization system to activate or deactivate individual sterilization element.

Yet another object of the present patent application is to use meteorological data to determine the most probable air speed within the air sterilization system due to local wind conditions; the computation system will control the sterilization system to provide sufficient amount of ultraviolet sterilization energy to deactivate pathogens while minimizing energy consumption.

Yet another object of the present patent application is to use sensors external to the device to capture wind speed data and provide said wind speed data to the computation system to ensure sufficient amount of ultraviolet sterilization energy is radiated to deactivate pathogens.

Yet another object of the present patent application is to combine sensor data and other meteorological conditions to form long term forecast of the sterilization energy requirements.

Yet another object of the present patent application is to ensure the air speed within the air sterilization system is reduced, by design and mechanical means, to ensure sufficient time for pathogen inactivation, while maintaining opening sizes in compliance with ordinances and regulations.

Yet another object of the present patent application is to ensure each individual sterilization element is utilized at the same rate; in addition to uniform utilization, based on the reported utilization, maintenance can be scheduled to replace the sterilization element before failure.

According to one embodiment of the present patent application, it provides an air sterilization system with remote monitoring and control. The remote monitoring and control system collects internal sterilization system data from temperature, intensity of radiation from sterilization elements, energy consumption, internal air speed and other relevant information. In addition to the internal system information, the monitoring and control system collect current meteorological information where the device is installed. External sensors for weather and meteorological information near the sterilization system can also transmit the information to the remote monitoring and control system.

The remote monitoring and control system, based on sensors and meteorological data, will determine the appropriate amount of sterilization energy required for inactivation of pathogens in the air coming out from the ventilating pipe. With the collected usage data from the sterilization system, the control system can select sterilization elements based on usage history or elements that are located at positions where replacement work can be done easier.

With the utilization data stored in the remote monitoring and control system, the system can predict when maintenance should be conducted and notify maintenance personnel to conduct periodic or emergency maintenance based on utilization and status of the sterilization system.

The present invention may be best understood and will become apparent from the following description with referencing to the accompanying drawings.

### BRIEF DESCRIPTION OF THE SEVERAL VIEWS OF THE DRAWING

FIG. 1 shows a perspective view of the air sterilization device 100 in accordance with the present invention, comprising of an upper connector, a lower connector and an enclosure;

FIG. 2 shows a bottom view of the air sterilization device 100;

FIG. 3 shows atop view of the air sterilization device 100;

FIG. 4 shows an internal view of the air sterilization device 100;

FIG. 5 shows a cross section view of the air sterilization device 100;

FIG. 6 shows the air sterilization device 100 mounted coaxially with the ventilating pipe;

FIG. 7 shows the air sterilization device 100 mounted coaxially with the ventilating pipe secured to wall structure;

FIG. 8 shows the sterilization device secured to wall structure with power connection and wireless connectivity;

FIG. 9 shows an overview of the components for the sterilization device;

FIG. 10a shows an exemplary setup with the air sterilization device and external sensors on a rooftop;

FIG. 10b shows another exemplary setup with a plurality of the air sterilization devices and external sensors on a rooftop;

FIG. 11 shows a process flow diagram in the remote monitoring and control system in determining the sterilization elements to activate based on input;

FIG. 12 shows a process flow diagram regarding a process in the remote monitoring and control system in selecting sterilization elements to optimize maintenance; and,

FIG. 13 shows a block diagram of a computing device.

### DETAILED DESCRIPTION OF THE INVENTION

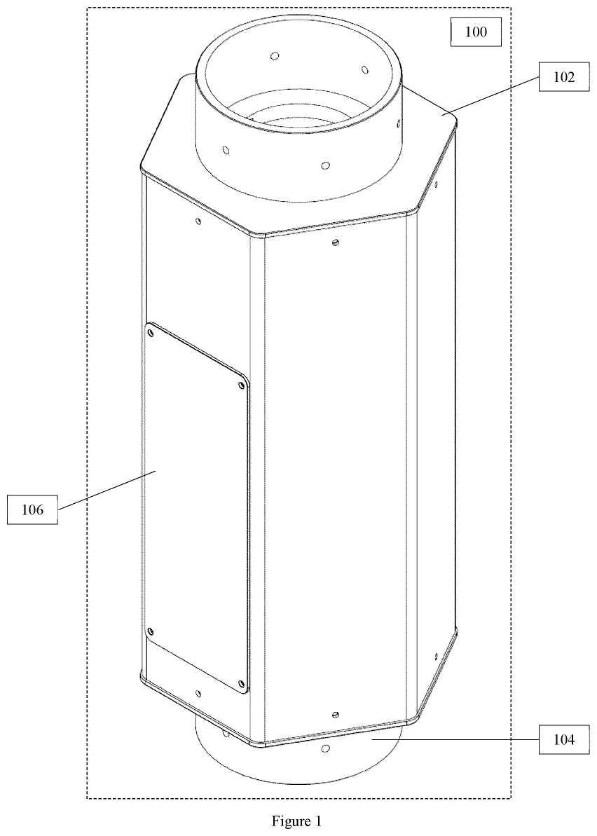

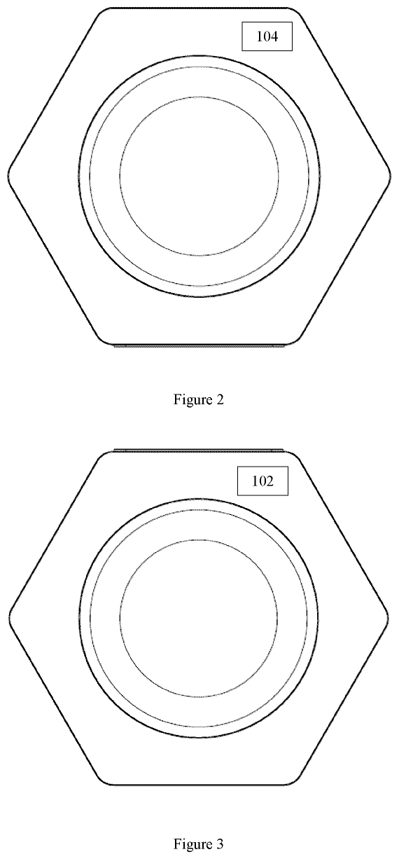

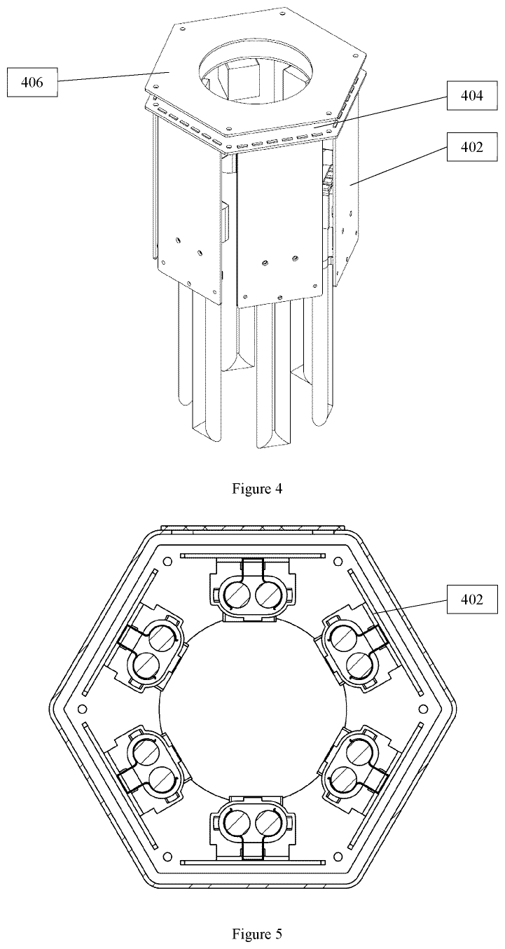

FIG. 1 shows a perspective view of the air sterilization device 100 in accordance with the present invention; FIG. 2 shows a bottom view of the air sterilization device 100 and, FIG. 3 shows a top view of the air sterilization device 100. Now, referring to FIG. 1, the air sterilization device 100 components consist of a housing, 103, a top connector 102 that interfaces with the upper segment of a ventilating pipe (not shown) being in communication with ambient air, a bottom connector 104 that interfaces with the lower segment of the ventilating pipe. A maintenance access panel 106 is provide on the housing 103 allows the sterilization elements (not shown) to be accessed for maintenance and replacement. Optionally, the top connector 102 may be in communication directly with the ambient air, without interfacing with a further ventilation pipe. Further optionally, the ventilation pipe for interfacing with the ambient air may be integrally formed and extending from the top connector 102.

FIG. 4 shows an internal view of the air sterilization device 100 (the housing 103 is removed). Controllable sterilization elements 402 are mounted radially within the sterilization device 100, and are defining a sterilization space 410. The sterilization elements 402 are connected to a control circuit board 404, where power controller and sensors for the sterilization elements are placed. The control circuit board 404 also communicates with the communication and process circuit board 406 to manage the wireless communication and control the sterilization elements 402 in sterilization device 100. FIG. 5 shows the cross-sectional view with six sterilization elements 402. According to a preferred embodiment of the present invention, the sterilization elements 402 are ultraviolet-C (or UV-C) radiation emitters, which emit ultraviolet light with a wavelength(s) between 200 nm and 280 nm. The number of the sterilization elements may be more than or less than six, as long as the sterilization element(s) provide sufficient sterilization effect on the volume of the air flowing through the sterilization space. In this regard, the sterilization elements of the present invention are configured to provide sufficient UV-C radiation at anywhere between about 2,000 and 8,000 (or higher) μW˜s/cm² for achieving about 90% kill of the most bacteria and viruses.

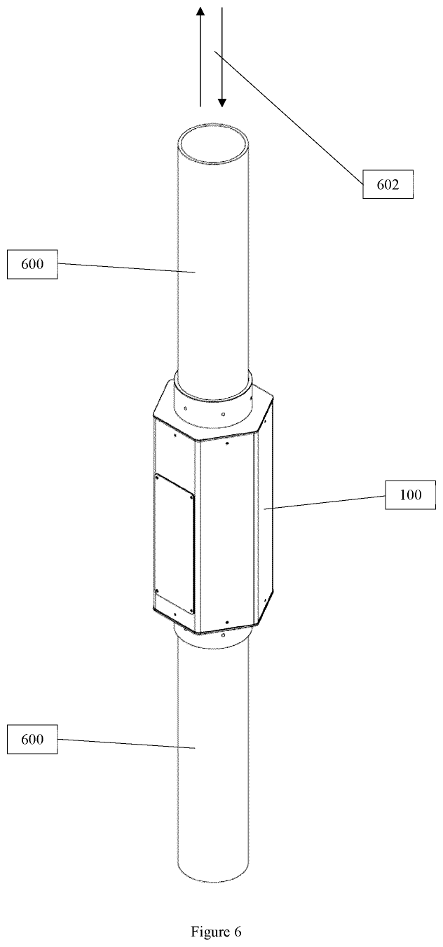

FIG. 6 shows an exemplary configuration of the air sterilization device 100 being installed co-axially aligned with plumbing ventilating pipe 600. During normal plumbing operations, air for anti-siphonage flows in or out 602 through at the top end/opening 604 of the plumbing ventilating pipe 600 to where air movement through the plumbing ventilating pipe 600 is needed to balance the air pressure(s) within the plumbing system (not shown) in the building. When the plumbing system is not in used and with wind blowing across the top end 604 of the ventilating pipe, negative pressure is developed at the top end 604 of the ventilating pipe 600. The negative pressure subsequently causes air in the ventilating pipe 600 to move up and out from the top end 604 of the ventilating pipe 600. The sterilization device 100 with the energized sterilization elements 402 ensures the escaping air from the ventilating pipe 600 is sterilized of pathogens before releasing into the environment.

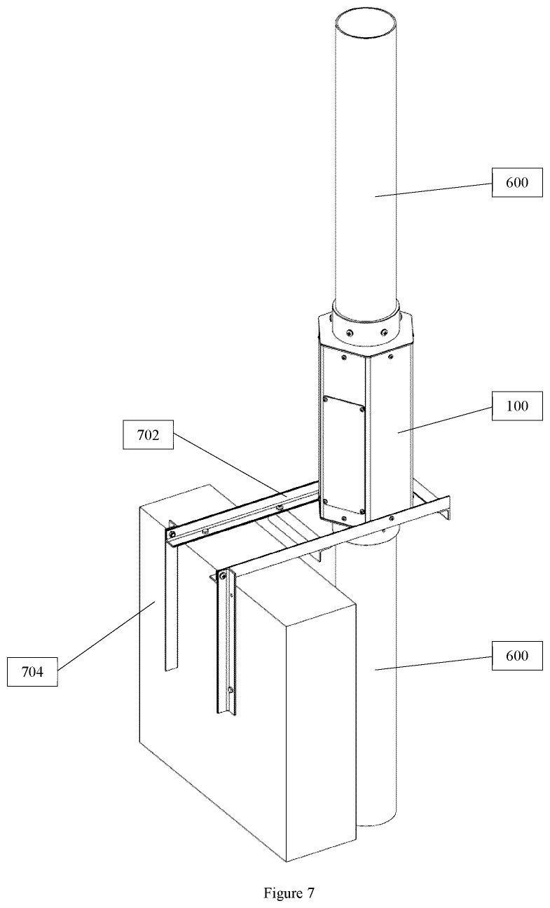

FIG. 7 shows another exemplary configuration of the air sterilization device 100. The air sterilization device 100 is installed co-axially with the ventilating pipe 600, at near the top end section thereof, and secured to the roof parapet wall 704 with a mounting frame 702. This configuration is to ensure the structural stability of the configuration/installation.

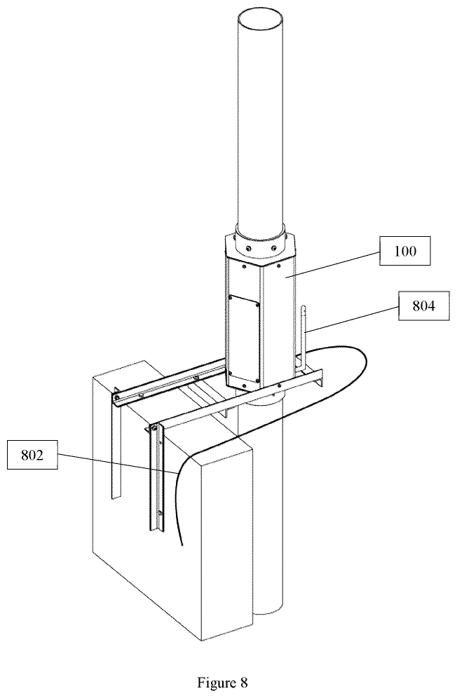

FIG. 8 shows yet another exemplary configuration of the air sterilization device 100 with the power connection 802 and wireless communication 804. The mounting frame 702 is secured to the roof parapet wall 704.

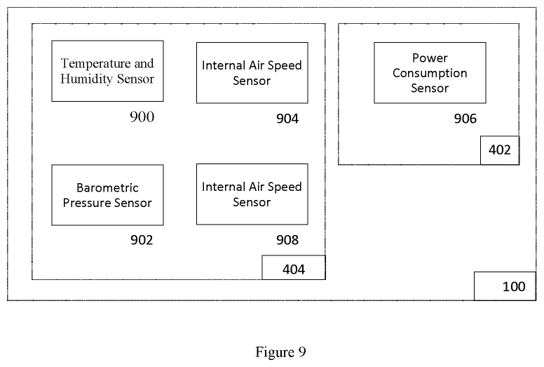

FIG. 9 shows an overview of sensors that provides information to the air sterilization device 100, and uses measurements to optimize the operation of the sterilization elements 402. Temperature and humidity sensors 900 provide temperature and humidity within the air passing through the air sterilization device 100. Barometric sensor 902 provides air pressure within the air sterilization device 100. Air speed sensors 904 provides air movement speed within the air sterilization device 100. Power consumption sensors 906 monitors power consumption on each of the sterilization elements 402. Sterilization power intensity sensors 908 are also utilized to monitor the sterilization power provided by the sterilization device 100 for pathogens inactivation.

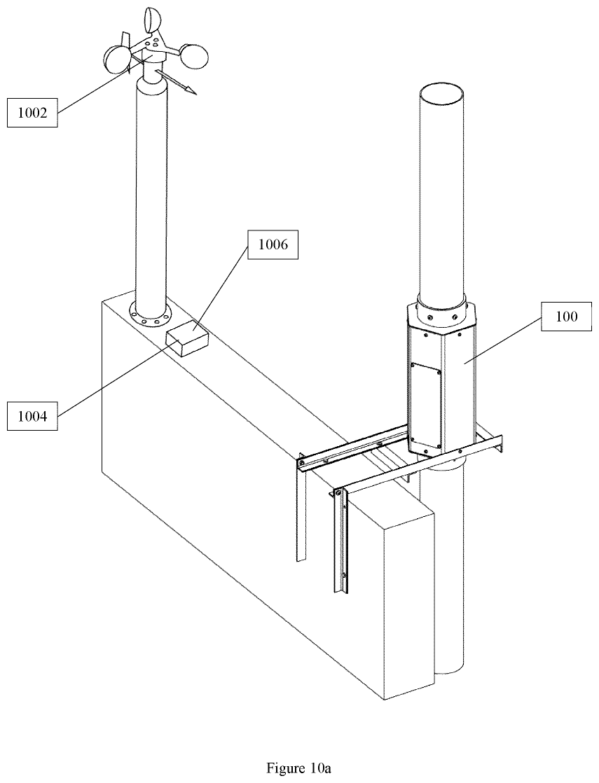

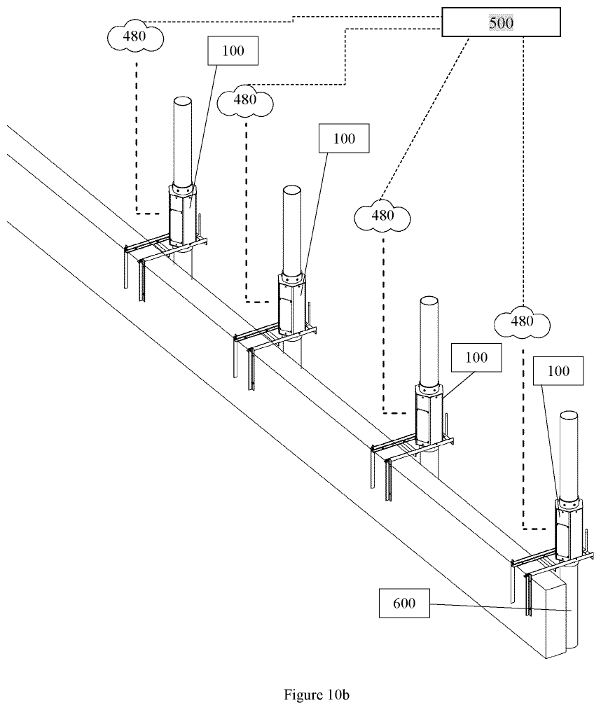

FIG. 10a shows a further exemplary configuration of the air sterilization device 100 with external wind speed and direction sensors 1002; external environmental barometric sensors 1004 that monitors barometric pressures in the outside environment; and, temperature and humidity sensors 1006 that monitor the external environmental conditions next to the air sterilization device. The barometric sensor 1004 and temperature and humidity sensors 1006 can be mounted within the same enclosure of the air sterilization device 100. The barometric sensor 1004 and temperature and humidity sensors 1006 can be an integrated sensor device.

As previously stated, the air sterilization devices 100 can be connected via a network (via wired and/or wireless network(s)). A number of air sterilization devices 100 may be remotely monitored and controlled by one or more remote monitoring and control systems 1100.

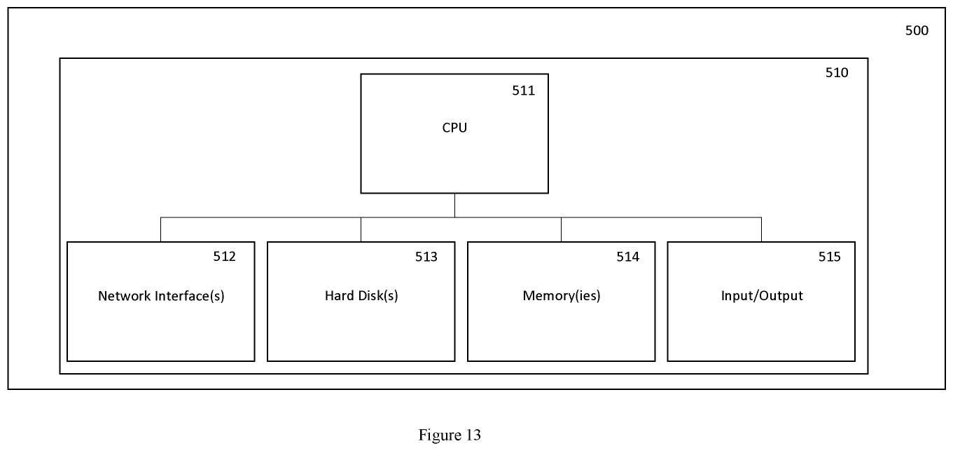

FIG. 10b shows an exemplary configuration of one or more of the air sterilization devices 100, connected to the remote monitoring and control system 500 through/via a wired or wireless connection(s) or network 480. The remote monitoring and control system 500 may comprises one or more computing devices 510 (shown in FIG. 13). Now, referring to FIG. 13, the computing device 510 comprises a central processing unit (or CPU) 511 for executing instructions; and, comprising a network interface(s) 512, a hard disk(s) or non-volatile data storage (such as FLASH or solid state data storage) 513, a memory(ies) 514, and input/output interface 515, all of which are in communication with CPU 511. CPU 511 executes the executable instructions stored in the hard disk(s) 513 to carry out the steps of the process required for remote monitoring and controlling the air sterilization devices 100. The computing device 510 may be a personal computer, laptop computer, handheld computing device, or smart phone. The computing device 510 may be a cloud computing platform, for example, Amazon Web Services (or AWS), that may provide required computing environment/features for realizing features/functionalities required for the remote monitoring and control system 500.

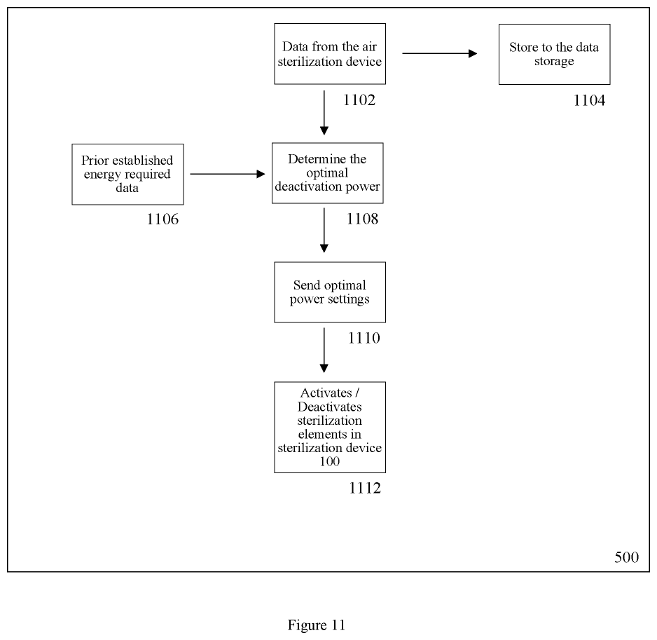

FIG. 11 shows a flow diagram of the remote monitoring and control system 500. The remote monitoring and control system 500 carries out a method consists of various process steps to manage the sterilization elements 402 within the air sterilization devices 100 through/via a wired or wireless connection(s) or network 480. At process step 1102, the remote monitoring and control system 500 receives measured/sensors data from the air sterilization device 100 which are transmitted and encrypted via communication circuit board 406 of the air sterilization device 100. The sensors data received at step 1102 are stored in a data storage (for example, a hard disk(s) 513) at step 1104. The remote monitoring and control system 500 retrieves priorly established energy requirement data from the data storage (for example, a hard disk(s) 513) at step 1106. The priorly established energy requirement data may be a calibration data measured and stored at the time of installation of each air sterilization device 100 or at the time of maintenance. Then, based on the sensors data received at step 1102 with the priorly established energy requirement data at step 1106, the remote monitoring and control system 500 determines the optimal power for sterilization at each of the air sterilization devices 100 to ensure that sufficient UV-C radiation, anywhere between about 2,000 and 8,000 (or higher) μW·s/cm² for achieving about 90% or higher kill of the most bacteria and viruses. At step 1110, the determined optimal power settings are sent back to the air sterilization devices 100 via the communication circuit 406. Each of the air sterilization devices 100 based on the instruction from the remote monitoring and control system 500 then controls the sterilization elements 402.

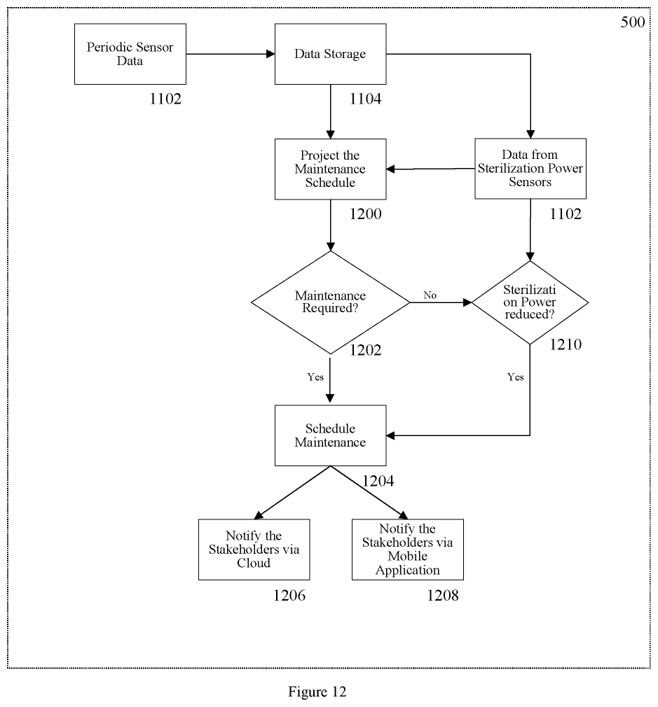

FIG. 12 shows an exemplary flow diagram 1200 of the remote monitoring and control system 500 for processing maintenance and optimization of utilization of the air sterilization device(s) 100. The remote monitoring and control system 500 receives periodic sensor data (for example, UV-C radiation sensor data and power consumption sensor data) at step 1201, which are sent from the air sterilization devices 100. The remote monitoring and control system 500 stores the data at step 1202. At step 1203, the remote monitoring and control system 500 further receives the real time monitored data from sterilization power sensors 906, the remote monitoring and control system 500 determines a projected maintenance schedule 1204 of the sterilization elements 402. The projected maintenance schedule may be pre-determined based on expected decay or efficiencies in UV-C radiation emission from UV-C radiation emitters and their projected/expected power consumptions over time. Based on the projected maintenance schedule 1204, current time and other factors such as general environmental trends, the remote monitoring and control system 500 determines whether maintenance is required at step 1205. Such decision may be made based on an artificial intelligence algorithm/AI model by training such AI model using a training data set. If maintenance is required at step 1205, a timing for maintenance is scheduled at step 1207. Notifications of maintenance programme can be sent to the maintenance personnel and stakeholders via cloud applications at 1208 or mobile applications at 1209. Even if it determines that maintenance is not required at step 1205, should monitored sensors data received at step 1203 indicate, for example, a certain reduction in sterilization power or reduction in power consumption at step 1206, the remote monitoring and control system 500 will ensure to schedule maintenance at step 1207 and notify maintenance personnel via cloud and mobile applications at steps 1208 and 1209.

1. An air sterilization device, comprising:
a. a housing having a first opening and second opening, and the first opening is adapted to interface with a plumbing system of a building and the second opening being in communication with ambient air;
b. a plurality of sterilization elements being enclosed in the housing, the plurality of sterilization elements defining an air passage that is in communication with the first opening and second opening; and,
c. a controller for controlling the plurality of the sterilization elements for sterilizing air passing through the air passage between the first opening and the second opening.

2. The air sterilization device as recited in claim 1, wherein the second opening of the housing is in communication with the ambient air via a pipe.

3. The air sterilization device as recited in claim 1 or 2, wherein the plurality of sterilization elements comprising ultraviolet-C (or UV-C) radiation emitters.

4. The air sterilization device as recited in any one of claims 1 to 3, wherein the plurality of sterilization elements comprising plasma/ion generators.

5. The air sterilization device as recited in claim 3 or 4 further comprising an internal air speed sensor being in communication with the controller for measuring air speed passing through the air passage for controlling the plurality of the sterilization elements to optimize UV-C radiation therefrom.

6. The air sterilization device as recited in claim 5 further comprising a barometric pressure sensor being in communication with the controller for measuring barometric pressure in the air passage for controlling the plurality of the sterilization elements.

7. The air sterilization device as recited in claim 6 further comprising a sterilization power intensity sensor being in communication with the controller for measuring the power intensity emitted from the plurality of the sterilization elements for controlling the plurality of the sterilization elements.

8. The air sterilization device as recited in claim 7, wherein the controller comprising a communication device for remotely communicating with a remote controller.

9. A air sterilization system, comprising:
a. a plurality of the air sterilization devices as recited in claim 8; and
b. the remote controller as recited in claim 8.
